(19) United States
(12) Patent Application Publication
(10) Pub. No.: US 2025/0332076 A1
(43) Pub. Date: Oct. 30, 2025

(54) STANNOUS CHLORIDE COMPOSITION FLOWABLE AT AMBIENT CONDITIONS

(71) Applicant: HONEYWELL INTERNATIONAL INC., Charlotte, NC (US)

(72) Inventors: Christian Werner, Hannover (DE); Elisabeth Memmel, Seelze (DE); Anke Zenker, Rinteln (DE)

(73) Assignee: HONEYWELL INTERNATIONAL INC., Charlotte, NC (US)

(21) Appl. No.: 19/073,056

(22) Filed: Mar. 7, 2025

Related U.S. Application Data
(60) Provisional application No. 63/639,076, filed on Apr. 26, 2024.

(51) Int. Cl. A61K 8/20; A61K 8/25; A61Q 11/00

(52) U.S. Cl. CPC A61K 8/20; A61K 8/25; A61Q 11/00

(57) ABSTRACT

A stannous chloride composition and method of making a composition that is flowable at ambient conditions and is substantially free of an anti-caking agent. The composition includes anhydrous stannous chloride, stannous chloride hydrate, and water in an amount of about 1 to about 10 weight percent based on a total weight of the composition. The anhydrous stannous chloride and the stannous chloride hydrate are present in a weight ratio of about 5:95 to about 81:19, respectively, and the composition has a dump angle of from about 20 to about 80 degrees.

### CROSS-REFERENCE TO RELATED APPLICATION

This application claims the benefit of U.S. Provisional application No. 63/639,076, filed Apr. 26, 2024, the entire contents of which are hereby incorporated by reference.

### TECHNICAL FIELD

The present disclosure generally relates to a stannous chloride composition that is flowable, and typically non-hydroscopic, at ambient conditions. More specifically, this disclosure relates to a stannous chloride composition that includes anhydrous stannous chloride, stannous chloride hydrate, and water present in a particular amount.

### BACKGROUND

Stannous chloride has been an important ingredient in oral care compositions for many years. Stannous chloride can provide antimicrobial effects, can control breath malodor, can control dental plaque growth and metabolism, can reduce gingivitis, can decrease progression of periodontal disease, can reduce dentinal hypersensitivity, and can reduce coronal and root dental caries and erosion.

However, incorporation of stannous chloride into many oral care compositions can be difficult. Stannous (II) chloride, both dihydrate and anhydrous forms, are hygroscopic and thus attract water vapor from the air through both absorption and adsorption. This leads to caking which can be described as particles binding together and forming agglomerates. This greatly reduces flowability and usefulness in commercial processes.

More specifically, shipping, storing, and handling stannous chloride compositions tends to be difficult due to the caking of the composition. This decreases the efficiency of removing the stannous chloride from storage facilities. In fact, even simple mass flow designs for removing the compositions from storage facilities tend to fail. For example, both the Peschl Shear Tester and bin design calculations indicate that the wall angles of storage bins need to be near vertical with a large outlet dimension to allow stannous chloride compositions to be removed.

To solve this problem, anti-caking agents have been used to improve the flow, decrease the compaction and therefore decrease flow restrictions of the stannous chloride compositions during processing. Anti-caking agents function either by adsorbing excess moisture or by coating particles to make them less prone to water adsorption.

US20100310614 discloses a composition including stannous chloride combined with silica as an anti-caking agent. Although the silica may decrease caking and improve flow, the addition of silica is an extra step in production which leads to higher costs and longer production times. Moreover, the inclusion of silica in oral care compositions can lead to its own unique drawbacks.

Accordingly, there remains an opportunity to develop a stannous chloride composition that is substantially free of an anti-caking agent but that remains flowable at ambient conditions. Furthermore, other desirable features and characteristics of the present disclosure will become apparent from the subsequent detailed description of the disclosure and the appended claims, taken in conjunction with the accompanying drawings and this background of the disclosure.

### SUMMARY

This disclosure provides a stannous chloride composition that is flowable at ambient conditions and is substantially free of an anti-caking agent. The stannous chloride composition includes anhydrous stannous chloride, stannous chloride hydrate, and water present in an amount of about 1 to about 10 weight percent based on a total weight of the stannous chloride composition. The anhydrous stannous chloride and the stannous chloride hydrate are present in a weight ratio of about 5:95 to about 81:19, respectively, and the stannous chloride composition has a dump angle of from about 20 to about 80 degrees. In one embodiment, the stannous chloride composition consists essentially of the anhydrous stannous chloride, the stannous chloride hydrate, and the water.

This disclosure also provides a method of forming the stannous chloride composition. The method includes the steps of providing the stannous chloride hydrate and drying the stannous chloride hydrate under vacuum to a water content of from about 1 to about 10 weight percent thereby forming anhydrous stannous chloride and the stannous chloride composition.

### BRIEF DESCRIPTION OF THE DRAWINGS

The present disclosure will hereinafter be described in conjunction with the following figures, wherein:

FIG. 1 is a photograph of a stannous chloride composition of the Examples that is not flowable and that exhibits caking, thereby representing the prior art;

FIG. 2 is a photograph of a stannous chloride composition of the Examples that is flowable and that exhibits a dump angle of about 30 degrees; and

FIG. 3 is a photograph of an additional stannous chloride composition of the Examples that is flowable and that exhibits a dump angle of about 60 degrees.

### DETAILED DESCRIPTION

The following detailed description is merely exemplary in nature and is not intended to limit the disclosure. Furthermore, there is no intention to be bound by any theory presented in the preceding background or the following detailed description.

Embodiments of the present disclosure are generally directed to stannous chloride compositions and methods for forming the same. For the sake of brevity, conventional techniques related to formation of such stannous chloride compositions may not be described in detail herein. Moreover, the various tasks and process steps described herein may be incorporated into a more comprehensive procedure or process having additional steps or functionality not described in detail herein. In particular, various steps in the manufacture of stannous chloride itself are well-known and so, in the interest of brevity, many conventional steps will only be mentioned briefly herein or will be omitted entirely without providing the well-known process details.

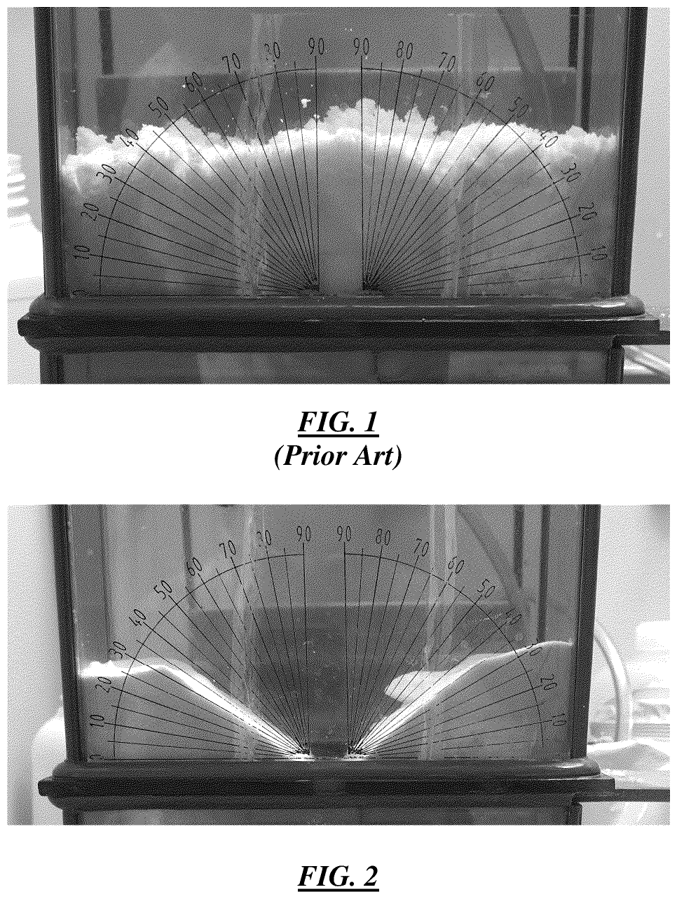

This disclosure provides a stannous chloride composition that is flowable at ambient conditions. The terminology “flowable” typically describes that the stannous chloride composition has a tipping angle or dump angle of from about 20 to about 80 degrees. In various embodiments, this angle is from about 25 to about 75, about 30 to about 65, about 35 to about 60, about 40 to about 55, or about 45 to about 50, degrees. The terminology “tipping angle” or “dump angle” typically describes an angle where materials will slide out of a tipped body, as is appreciated by one of skill in the art. Relative to this disclosure, the tipping angle or dump angle can be visually determined using a method known in the art as “according to Kawecki.” More specifically, this determination is performed visually using the apparatus as shown in FIGS. 1-3. Typically, this determination is made at ambient conditions, e.g. at room temperature (25° C.±3° C.) and at a relative humidity of about 40%+5%. In various non-limiting embodiments, all values and ranges of values including and between those set forth above are hereby expressly contemplated for use herein.

Alternatively, the terminology “flowable” may describe the stannous chloride composition being in a non-caked, agglomerated, or stuck-together form. Typically, a flowable stannous chloride composition is a stannous chloride composition wherein particles are not adhered to each other or otherwise held together by weak physical interactions. The “flowability” of a stannous chloride composition may be as determined by one of skill in the art.

The stannous chloride composition is typically substantially free of an anti-caking agent. For example, the stannous chloride composition may include less than about 5, 4, 3, 2, 1, 0.5, or 0.1, weight percent of the anti-caking agent based on a weight of the stannous chloride composition. The stannous chloride composition may alternatively be completely free of the anti-caking agent. The anti-caking agent may be any known in the art. For example, the anti-caking agent may be silica, tricalcium phosphate, powdered cellulose, magnesium stearate, sodium bicarbonate, sodium ferrocyanide, potassium ferrocyanide, calcium ferrocyanide, bone phosphate (i.e. calcium phosphate), sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane, and/or combinations thereof. In one embodiment, the stannous chloride composition is free of, or substantially free of, silica. In other embodiments, the stannous chloride composition is free of, or substantially free of, or includes less than about 5, 4, 3, 2, 1, 0.5, or 0.1, weight percent of, one or more additives known in the art, such as silica (silicon dioxide). In various non-limiting embodiments, all values and ranges of values including and between those set forth above are hereby expressly contemplated for use herein.

The stannous chloride composition includes anhydrous stannous chloride and stannous chloride hydrate. Stannous chloride is also known as tin (II) chloride and has the formula SnCl₂. The terminology “anhydrous” typically describes that the stannous chloride includes less than 1, 0.5, 0.1, 0.05, or 0.01, weight percent of water based on a total weight of the stannous chloride. Alternatively, the anhydrous stannous chloride may be free of, or substantially free of, water, i.e., dry. In various non-limiting embodiments, all values and ranges of values including and between those set forth above are hereby expressly contemplated for use herein.

Relative to the stannous chloride hydrate, this compound is typically described as a dihydrate (i.e., SnCl₂·2H₂O). However, some molecules may have more or less than two water molecules. As such, the hydrate, as a whole, may include an average of about two or more or less than about two, water molecules. For example, various molecules may have the formulae as follows: Sn₂Cl₄·3H₂O or SnCl₂·2H₂O. The terminology “stannous chloride hydrate” may be a term that encompasses both the dihydrate and distannous tetrachloride tri-hydrate (Sn₂Cl₄·3H₂O) or only the dihydrate. Accordingly, the stannous chloride composition may include the distannous tetrachloride tri-hydrate as part of the stannous chloride hydrate.

The anhydrous stannous chloride can be supplied in various forms: powder, flake and pellets. Stannous chloride dihydrate is commercially available from various suppliers. Physical characteristics include a colorless crystalline material with a slight characteristic odor, poor flow characteristics and a relatively short shelf life. However, the disadvantages of using stannous chloride, either in the dihydrate or anhydrous form, are the manufacturing constraints, both in shipping and handling. Both the dihydrate and the different anhydrous forms, to a greater or lesser extent, are hygroscopic, making flowability during processing difficult and giving a poor activity over the shelf life of the material. Stannous chloride is also an aggressive reducing agent to some metals. This property can lead to the formation of undesirable compounds because the material cakes and sits on dead spots in storage bins. However, it has been surprisingly found that the presence of water in an amount of from about 1 to about 10 weight percent based on a total weight of the stannous chloride composition allows the stannous chloride composition to be flowable at ambient conditions, as is first introduced above and described in greater detail below.

Moreover, in this disclosure, the anhydrous stannous chloride and the stannous chloride hydrate may be supplied independently from one another or the anhydrous stannous chloride may be formed from the stannous chloride hydrate such that the anhydrous stannous chloride is formed in-situ from the stannous chloride hydrate, e.g. as described below relative to the method of this disclosure.

The anhydrous stannous chloride and the stannous chloride hydrate are present in a weight ratio of from about 5:95 to about 81:19. In various embodiments, this weight ratio is about 10:90, about 15:85, about 20:80, about 25:75, about 30:70, about 35:65, about 40:60, about 45:55, about 50:50, about 55:45, about 60:40, about 65:35, about 70:30, about 75:25, or about 80:20, respectively, or any range therebetween. The anhydrous stannous chloride and the stannous chloride hydrate are also typically present in a molar ratio of from about 1:10 to about 10:1, respectively. In various embodiments, this weight ratio is about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 1:2, or about 1:1, respectively. In various non-limiting embodiments, all values and ranges of values including and between those set forth above are hereby expressly contemplated for use herein.

The stannous chloride composition also includes water present in an amount of about 1 to about 10 weight percent based on a total weight of the stannous chloride composition. The water typically originates from the stannous chloride hydrate (e.g. dihydrate). However, external water, such as water vapor absorbed from the atmosphere may contribute to the water of the stannous chloride composition as well. In various embodiments, the amount of water is about: 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, or 10, weight percent based on a total weight of the stannous chloride composition. In other embodiments, the amount of water is from about 2 to about 8, about 3 to about 9, about 4 to about 8, about 5 to about 7, about 5 to about 6, or about 4 to about 8, weight percent based on a total weight of the stannous chloride composition. In various non-limiting embodiments, all values and ranges of values including and between those set forth above are hereby expressly contemplated for use herein.

In various embodiments, the stannous chloride composition includes the anhydrous stannous chloride, the stannous chloride hydrate, and the water. In other embodiments, the stannous chloride composition includes the anhydrous stannous chloride, the stannous chloride hydrate (e.g. dihydrate and/or distannous tetrachloride tri-hydrate), and the water. In additional embodiments, the stannous chloride composition consists essentially of the anhydrous stannous chloride, the stannous chloride hydrate, and the water. In other embodiments, the stannous chloride composition consists essentially of the anhydrous stannous chloride, the stannous chloride hydrate (e.g. dihydrate and/or distannous tetrachloride tri-hydrate), and the water. In further embodiments, the stannous chloride composition consists of the anhydrous stannous chloride, the stannous chloride hydrate, and the water. In other embodiments, the stannous chloride composition consists of the anhydrous stannous chloride, the stannous chloride hydrate (e.g. dihydrate and/or distannous tetrachloride tri-hydrate), and the water.

In various embodiments, the stannous chloride composition is non-hygroscopic. For example, the stannous chloride composition may adsorb less than 0.1, 0.2, 0.3, 0.4, 0.5, 0.6, 0.7, 0.8, 0.9, or 1.0, wt % of water vapor from the atmosphere when exposed to ambient conditions (e.g. at room temperature (25° C.±3° C.) and at a relative humidity of about 40%+5%) for about 20 hours.

### Method of Forming the Stannous Chloride Composition:

This disclosure also provides a method of forming the stannous chloride composition. The method includes the step of providing the stannous chloride hydrate. This step can be further defined as supplying or making available a source of stannous chloride hydrate for the method. The method also includes the step of drying the stannous chloride hydrate under vacuum. The vacuum pressure can be any known in the art. Alternatively, it is possible to dry the stannous chloride hydrate without a vacuum. However, drying times might be longer. The stannous chloride hydrate can be dried at ambient temperature or at elevated temperatures, e.g. at temperatures as high as those approaching the melting point of the stannous chloride hydrate. Typically, the stannous chloride hydrate is dried at a temperature of from about 25° C. to about 100° C., from about 40° C. to about 90° C., or from about 50° C. to about 80° C. The temperature can be ramped up at any speed. However, the temperature is typically ramped at a rate of about 10° C./hr. to about 200° C./hr., or about 20° C./hr. to about 160° C./hr., or about 80° C./hr. to about 100° C./hr. The ramping of the temperature aids in drying the stannous chloride hydrate to a desired water content. In various non-limiting embodiments, all values and ranges of values including and between those set forth above are hereby expressly contemplated for use herein.

In fact, the stannous chloride hydrate is typically dried to a water content of from about 1 to about 10 weight percent thereby forming anhydrous stannous chloride and the stannous chloride composition. In various embodiments, this water content is about 1.5, about 2, about 2.5, about 3, about 3.5, about 4, about 4.5, about 5, about 5.5., about 6, about 6.5, about 7, or about 7.5, weight percent based on a total weight of the stannous chloride hydrate. The drying of the stannous chloride hydrate removes some of the water from the hydrate thereby forming the anhydrous stannous chloride. Typically, the drying is stopped once the desired water content is reached. The specific time of drying is not limited and may be any chosen by one of skill in the art considering, for example, the strength of the vacuum, the temperature of heating, etc. After drying, the stannous chloride composition is typically crystalline and has the dump angle as is descried above. The stannous chloride composition can then be utilized to form other compositions such as the oral care composition described in greater detail below. In various non-limiting embodiments, all values and ranges of values including and between those set forth above are hereby expressly contemplated for use herein.

### Oral Care Composition:

This disclosure also provides an oral care composition that includes the aforementioned stannous chloride composition. The oral care composition is not particularly limited and may be further described as a toothpaste, gel, mouthwash, lozenge, gum, dentifrice, etc. The oral care composition may include any one or more additives, compounds, etc. known in the art.

In various embodiments, the oral care composition includes a water-soluble fluoride compound in an amount sufficient to give a fluoride ion concentration sufficient to provide anticaries effectiveness. For example, the oral care composition may include a fluoride ion source sufficient to provide from about 0.01% to about 0.35% (about 100 to about 3500 ppm), typically from about 0.03% to about 0.2% by weight of the oral care composition (300 to 2000 ppm) fluoride ions. A wide variety of fluoride ion-yielding materials can be employed as sources of soluble fluoride such as stannous fluoride, sodium fluoride, potassium fluoride, sodium monofluorophosphate, indium fluoride and many others. Typical sources of fluoride ion are stannous fluoride and sodium fluoride, as well as mixtures thereof. In various non-limiting embodiments, all values and ranges of values including and between those set forth above are hereby expressly contemplated for use herein.

In other embodiments, the oral care composition includes a dental abrasive to remove surface stains and for polishing the teeth. Dental abrasives suitable for use include insoluble sodium polymetaphosphate, hydrated alumina, and resinous abrasive materials such as particulate condensation products of urea and formaldehyde. Particulate thermo-setting polymerized resins, for example, melamines, phenolics, ureas, melamine-ureas, melamine-formaldehydes, urea-formaldehyde, melamine-urea-formaldehydes, cross-linked epoxides, cross-linked polyesters, and combinations thereof.

In further embodiments, the oral care composition includes a chelant, e.g. one having an average molecular weight of less than about 500, 1,000, or 1,500 g/mol. The term “chelant”, as used herein typically describes a bi- or multidentate ligand having at least two groups capable of binding to divalent metal ions. In various non-limiting embodiments, all values and ranges of values including and between those set forth above are hereby expressly contemplated for use herein.

In still other embodiments, the oral care composition includes a humectant. The humectant contributes to the oral care composition maintaining softness upon exposure to air, to give a moist feel to the mouth, and, for particular humectants, to impart a desirable sweetness of flavor. The humectant, on a pure humectant basis, generally includes from about 5% to about 70%, typically from 15% to 45%, by weight of the oral care composition. Suitable humectants include edible polyhydric alcohols such as glycerin, sorbitol, xylitol, butylene glycol, polyethylene glycol, and propylene glycol, especially sorbitol and glycerin. In various non-limiting embodiments, all values and ranges of values including and between those set forth above are hereby expressly contemplated for use herein.

In still other embodiments, the oral care composition includes a surfactant. The surfactant is not particularly limited and may be an anionic, nonionic, cationic, or betaine surfactant, or a combination thereof. Anionic surfactants can be included to provide cleaning and foaming properties, and are typically used in an amount from about 0.1% to about 2.5%, or from about 0.3% to about 2.5% or from about 0.5% to about 2.0% by weight of the oral care composition. In various non-limiting embodiments, all values and ranges of values including and between those set forth above are hereby expressly contemplated for use herein.

In further embodiments, the oral care composition includes a thickening agent or binder to provide a desirable consistency, to provide desirable active release characteristics upon use, to provide shelf stability, and to provide stability of the composition, etc. Thickening agents can include carboxyvinyl polymers, carrageenan, nonionic cellulose derivatives such as hydroxyethyl cellulose (HEC), and water soluble salts of cellulose derivatives such as sodium carboxymethylcellulose (NaCMC). Natural gums such as gum karaya, xanthan gum, gum arabic, and gum tragacanth can also be used herein. Suitable thickening agent levels can range from about 0.1 to about 5% by weight of the oral care composition and higher if necessary. In various non-limiting embodiments, all values and ranges of values including and between those set forth above are hereby expressly contemplated for use herein.

In other embodiments, the oral care composition includes a desensitizing agent to control hypersensitivity, especially salts of potassium and strontium such as potassium nitrate. The oral care composition can also include an antimicrobial agent such as water insoluble non-cationic antimicrobial agents. These may include halogenated diphenyl ethers, particularly triclosan and essential oils such as thymol. Water soluble antimicrobials include quaternary ammonium salts such as cetyl pyridinium chloride. Enzymes are another type of active agent that may be used in the oral care composition. Inorganic antimicrobial agents can also be used. One such source is zinc ions. Preferred zinc sources are zinc chloride, zinc sulphate, zinc citrate, zinc gluconate, zinc lactate and zinc glycinate. Additional sources of stannous ions can also be incorporated. Suitable stannous sources include stannous fluoride, stannous acetate, stannous gluconate, stannous oxalate, stannous sulfate, stannous lactate and stannous tartrate.

It is also contemplated that the oral care composition may include flavoring and/or sweetening agents. Suitable flavoring agents and sweetening agents are well known in the art. Suitable flavor levels in the oral care compositions herein are from about 0.1% to about 5.0%, more typically from about 0.5% to about 1.5%, by weight of the oral care composition. Typically, a flavor oil will be manufactured in a separate step and will include multiple components, natural and/or synthetic in origin, in order to provide a balanced flavor which is acceptable to a broad range of people. Flavor components can be chosen from mint, spice, fruit, citrus, herbal, medicinal, and common food flavor types (e.g. chocolate). A physiological cooling agent can also be incorporated into the flavor oil. The coolant can be any of a wide variety of materials. Included among such materials are carboxamides, menthol, acetals, ketals, diols, and mixtures thereof. In various non-limiting embodiments, all values and ranges of values including and between those set forth above are hereby expressly contemplated for use herein.

Sweetening agents which can be used include sucrose, glucose, saccharin, sucralose, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate, sucralose and sodium saccharin, and mixtures thereof. The oral care composition can include from about 0.1% to about 3% by weight of these agents, more typically from about 0.1% to about 1%, by weight, based on a total weight of the oral care composition. In further embodiments, the oral care composition may include one or more pigments, dyes and opacifiers, such as titanium dioxide. In various non-limiting embodiments, all values and ranges of values including and between those set forth above are hereby expressly contemplated for use herein.

### Method of Forming the Oral Care Composition:

This disclosure also provides a method of forming the oral care composition. This method includes the step of providing the stannous chloride composition as is described above and combining the stannous chloride composition with one or more of the aforementioned components, e.g. abrasives, chelants, surfactants, etc., to form the oral care composition.

### EXAMPLES

A series of stannous chloride compositions are formed and evaluated to determine flowability/caking.

A first composition (Composition 1; Comparative) is formed as follows: Tin metal is dissolved in hydrochloric acid in the presence of oxygen from the air. The resulting solution includes stannous chloride dihydrate which is concentrated by water evaporation to crystallize the product and form a suspension. Solid stannous chloride dihydrate is then isolated by centrifugation of the suspension.

A second composition (Composition 2; Inventive) is formed as follows: The material formed above in Composition 1 is dried under vacuum using rotating movement. The temperature is ramped at a rate of about 10° C./h to about 80° C./h to a final temperature of about 80° C. to about 100° C. Without intending to be bound by any theory, it is believed that this form of rotating drying unexpectedly produces additional attrition, which may form smaller and rounder particles of the Composition thereby contributing to a decreased dump angle, which is desirable.

A third composition (Composition 3; Inventive) is formed as follows: The material formed above in Composition 1 is dried under vacuum in an oven without movement. The temperature is ramped at a rate of about 10° C. to about 200° C. to a final temperature of about 80° C. to about 100° C.

After formation, each of the Compositions 1-3 are visually evaluated to determine dump angle according to a method provided by Kawecki and utilizing the apparatus as shown in FIGS. 1-3. These determinations are made at ambient conditions, e.g. at room temperature (25° C.±3° C.) and at a relative humidity of about 40%±5%.

The results of the evaluation of Composition 1 are shown in FIG. 1 and represent the prior art. It is clear that Composition 1 exhibits caking and agglomeration. The dump angle is not considered to be measurable.

The results of the evaluation of Composition 2 are shown in FIG. 2. It is clear that Composition 2 is flowable and exhibits minimal caking and agglomeration. The dump angle is determined to be about 30 degrees.

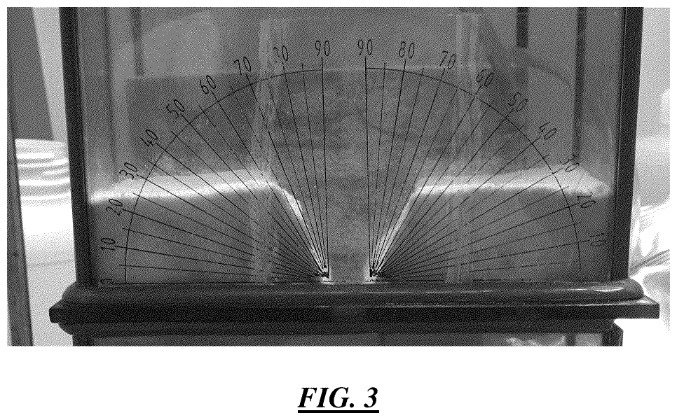

The results of the evaluation of Composition 3 are shown in FIG. 3. It is clear that Composition 3 is also flowable and exhibits minimized caking and agglomeration. The dump angle is determined to be about 60 degrees.

It has been surprisingly found that the presence of water in an amount of from about 1 to about 10 weight percent based on a total weight of the stannous chloride composition allows the stannous chloride composition to be flowable at ambient conditions. These results are surprising and unexpected. More specifically, it would be expected that the remaining water in crystals in Compositions 2 and 3 would cause the stannous chloride compositions to cake and absorb water from the environment due its hygroscopic behavior. Instead, only about 0.2% by weight of water is adsorbed over a period of about 20 hours when the Compositions 2 and 3 are exposed to ambient conditions. Furthermore, the flowability of the Compositions 2 and 3 is surprisingly unaffected.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A stannous chloride composition comprising:
a) anhydrous stannous chloride;
b) stannous chloride hydrate; and
c) water present in an amount of about 1 to about 10 weight percent based on a total weight of the stannous chloride composition,
wherein the stannous chloride composition is flowable at ambient conditions and contains less than about 5 wt. % of an anti-caking agent,
wherein the anhydrous stannous chloride and the stannous chloride hydrate are present in a weight ratio of about 5:95 to about 81:19, respectively, and
wherein the stannous chloride composition has a dump angle of from about 20 to about 80 degrees.

2. The stannous chloride composition of claim 1, wherein the composition is non-hygroscopic and absorbs less than 0.2 wt % of water when exposed to the atmosphere at about 25° C. and at a relative humidity of about 40% for about 20 hours.

3. The stannous chloride composition of claim 1, wherein the water is present in an amount of about 2 to about 8 weight percent based on a total weight of the stannous chloride composition.

4. The stannous chloride composition of claim 1, wherein the water is present in an amount of about 4 to about 8 weight percent based on a total weight of the stannous chloride composition.

5. The stannous chloride composition of claim 1, wherein the anhydrous stannous chloride and the stannous chloride hydrate are present in a weight ratio of about 30:70, respectively.

6. The stannous chloride composition of claim 1, having a dump angle of from about 35 to about 65 degrees.

7. The stannous chloride composition of claim 1, wherein the stannous chloride hydrate comprises stannous chloride dihydrate and distannous tetrachloride tri-hydrate.

8. The stannous chloride composition of claim 1, wherein the anti-caking agent is silica.

9. The stannous chloride composition of claim 1, further comprising a toothpaste.

10. A method of forming a stannous chloride composition comprising the steps of:
a. providing the stannous chloride hydrate; and
b. drying the stannous chloride hydrate under vacuum to a water content of from about 1 to about 10 weight percent thereby forming anhydrous stannous chloride and the stannous chloride composition;
wherein the composition is flowable at ambient conditions, contains less than about 5 wt. % of an anti-caking agent, and comprises anhydrous stannous chloride, stannous chloride hydrate, and water present in an amount of about 1 to about 10 weight percent based on a total weight of the composition,
wherein the anhydrous stannous chloride and the stannous chloride hydrate are present in a weight ratio of about 5:95 to about 81:19, respectively, and
wherein the composition has a dump angle of from about 20 to about 80 degrees, the method.

11. The method of claim 10, wherein the stannous chloride composition is non-hygroscopic and absorbs less than 0.2 wt % of water when exposed to the atmosphere at about 25° C. and at a relative humidity of about 40% for about 20 hours.

12. The method of claim 10, wherein the water is present in the stannous chloride composition in an amount of about 2 to about 8 weight percent based on a total weight of the stannous chloride composition.

13. The method of claim 12, wherein the water is present in the stannous chloride composition in an amount of about 4 to about 8 weight percent based on a total weight of the stannous chloride composition.

14. The method of claim 10, wherein the step of drying is further defined as drying the stannous chloride hydrate at a temperature of about 80° C. to a water content of from about 1 to about 10 weight percent.

15. The method of claim 10, wherein the anhydrous stannous chloride and the stannous chloride hydrate are present in the stannous chloride composition in a weight ratio of about 30:70, respectively.

16. The method of claim 10, wherein the stannous chloride hydrate comprises stannous chloride dihydrate and distannous tetrachloride tri-hydrate.

17. The method of claim 10, wherein the anti-caking agent is silica.
